(19) United States
(12) Patent Application Publication
(10) Pub. No.: US 2003/0199852 A1
(43) Pub. Date: Oct. 23, 2003

(54) Attachment joints with polymer encapsulation

(75) Inventors: Kirk Patrick Seward, Dublin, CA (US); Isidro M. Gandionco, Fremont, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ENDOBIONICS, INC., San Leandro, CA

(21) Appl. No.: 10/421,997

(22) Filed: Apr. 22, 2003

Related U.S. Application Data
(63) Continuation-in-part of application No. 10/393,700, filed on Mar. 19, 2003.
(60) Provisional application No. 60/375,252, filed on Apr. 23, 2002.

(51) Int. Cl. A61M 25/16; A61M 25/18; A61M 39/00; A61M 39/10

(52) U.S. Cl. 604/533; 427/207.1; 427/248.1; 604/164.13

(57) ABSTRACT

Tubular catheters having two or more axially adjacent segments are reinforced by encapsulation in a polymeric layer which provides enhanced tensile strength. Exemplary polymers include parylene, silicone, PTFE, PVDF and the like. The layers are preferably vapor coated to thicknesses of 100 μm or below. Specific rapid exchange and needle injection embodiments are illustrated.

### CROSS-REFERENCES TO RELATED APPLICATIONS

This application claims the benefit of provisional application No. 60/375,252 (Attorney Docket No. 021621-000500US) filed on Apr. 23, 2002, the full disclosure of which is hereby incorporated herein by reference. This application is also a continuation-in-part of application Ser. No. 10/393,700 (Attorney Docket No. 021621-001500US), filed on Mar. 19, 2003, the full disclosure of which is incorporated herein by reference.

### BACKGROUND OF THE INVENTION

1. Field of the Invention

The present invention relates generally to medical apparatus and methods for fabricating such apparatus. In particular, the present relates to catheters having attachment joints between successive components or portions thereof.

The use of “rapid exchange” catheters in interventional cardiology has become widespread. Rapid exchange catheters are characterized by relatively short guidewire lumens, permitting the use of a shorter guidewire which reduces the time necessary to remove a first catheter and exchange that catheter for another over a guidewire. A particular type of rapid exchange catheter is referred to as a “monorail” catheter. Monorail catheters have very short guidewire lumens, typically 5 cm or less, which are usually incorporated within a distal tip of the catheter.

Of particular interest to the present invention, such monorail guidewire tips are often formed as separate components which are joined to the remaining proximal portion of a catheter body using adhesives, ultrasonic welding, or the like. Usually, the distal tip will have different mechanical properties from the remainder of the catheter body, and the joint between the distal tip and the remaining catheter body is a high stress point as the catheter is introduced through tortuous regions of the vasculature. Thus, the attachment point between the distal tip and catheter body in a monorail catheter can be a failure point in the catheter construction.

While the joint between a catheter tip and catheter body may be reinforced in a variety of ways, at least most reinforcement techniques result in increasing the size and/or rigidity of the catheter construction. Increases in size and/or rigidity are undesirable, particularly for catheters which must be very small for intended use within the coronary vasculature.

For these reasons, it would be desirable to provide improved methods for attaching distal catheter tips to proximal catheter bodies in monorail and other catheter designs. It would be particularly advantageous if these methods were useful for the joining of any adjacent catheter components and optionally even other components, such as electronic or other surface components, of the catheter constructions. It is a particular objective of the present invention to provide methods and structures for enhancing the strength of joints between adjacent axial and other catheter components without significantly increasing the width and/or rigidity of the catheter. As a further objective that the methods be simple and economic to perform and be useful with a wide range of vascular and other medical catheters. At least some of these objectives will be met by the inventions described hereinafter.

2. Description of the Background Art

U.S. Pat. No. 6,187,130 B1 describes a catheter having a tubular member overlaying a catheter body and an adjacent tip. U.S. Pat. Nos. 6,503,223 B1; 5,836,306; 5,468,225; 5,443,457; 5,383,853 and 5,330,444 describe a number of specific rapid exchange catheter constructions.

### BRIEF SUMMARY OF THE INVENTION

In a first aspect of the present invention, a catheter comprises a tubular catheter body and an encapsulation layer. The tubular catheter body includes at least two axially adjacent segments which are composed of materials having different mechanical properties, typically having different hardnesses. The segments may be joined together in a variety of ways, such as a butt joint, a press-fit joint, or the like, and may optionally be further secured by an adhesive, such as cyanoacrylate, silicone, or by heat welding, ultrasonic welding, or the like.

The encapsulation layer is provided to strengthen the junction between the two or more axially adjacent segments. The encapsulation layer will have a tensile strength of at least about 10 MPa, usually at least 20 MPa, and preferably at least 40, MPa or higher. Such tensile strength is preferably achieved with a very thin layer, typically below 100 μm, usually below 50 μm, and preferably below 10 μm. Such thin encapsulation layers provide strong joints which are resistant to kinking and separation with minimal increase in catheter diameter.

Preferred encapsulation layers are formed from polymers, such as parylene and other polyparaxylylenes; silicones; polytetrafluoroethylenes (PTFE's); polyvinylidene fluoride (PVDF), and the like. Particularly preferred are vapor deposited polymers, most particularly being vapor deposited parylene, although others of the exemplary polymers may also be applied by dip coating, such as dip-coated silicones. Vapor deposited parylene layers over catheter bodies formed from porous elastomers, such as silicone, are particularly advantageous because they form a composite having a very high shear (bond) strength with a high resistance to delamination and joint failure.

In an exemplary embodiment, the segments of the tubular catheter body will comprise a tip segment and a proximal body segment. The tip segment will usually be softer (or in some cases harder) than the proximal body segment, and the junction between the two will be a point of high stress which is strengthened according to the present invention.

In a second aspect of the present invention, a rapid exchange catheter comprises a catheter body and a distal tip having a guidewire lumen of 5 cm or less. The tip is attached to a distal end of the catheter body, typically using a butt joint or a press-fitted joint and optionally being further secured using an adhesive, adhesive welding, ultrasonic welding, or the like. Other preferred aspects of the rapid exchange catheter are generally as described above in connection with the first embodiment of the catheter of the present invention. Additionally, however, the vapor deposition of parylene and other lubricious polymers will advantageously be applied to the surface of the guidewire lumen as well. Such a lubricious coating facilitates introduction of the catheter over a guidewire, further relieving stress between the catheter tip and the remainder of the catheter body as the catheter is introduced over a guidewire.

In a third aspect of the present invention, a needle injection catheter comprises a catheter body having a proximal end and a distal end. A needle is reciprocatably disposed in the catheter body near its distal end, and a guidewire lumen is disposed in the distal end of the catheter body distally of the needle. Usually the needle advances outwardly through the catheter body in a first radial direction or the guidewire lumen entry port on the side of the catheter body is circumferentially spaced-apart from the exit port of the needle, preferably being offset by at least 90°, and more preferably being offset by at least 135°, typically being offset by about 180°.

In the specific embodiments, the catheter body of the needle injection catheter will comprise at least a proximal body portion in the distal tip, where the body portion and tip are usually composed of materials having different hardnesses or other mechanical properties and are joined at a junction region. The junction region is typically encapsulated in an encapsulation layer, as described above, which strengthens the joint between the tip and the remainder of the catheter body. Still further preferably, the distal tip will have a guidewire lumen form therein. The remaining aspects of this third embodiment are generally as described above in connection with the prior two embodiments of the present invention.

In a fourth aspect of the present invention, a method for fabricating an elongate catheter having at least two adjacent segments composed of polymeric materials having different mechanical properties comprises encapsulating a joint region with a layer of material having a tensile strength in the axial direction of at least 10 MPa. The joint region is disposed at a location where the two axially adjacent segments meet, and the encapsulation step preferably comprises vapor depositing a polymer layer over the catheter body at said joint region, often being over at least one-half the total length of the catheter, and typically being over substantially the entire length of the catheter. The nature of the polymers, thicknesses of depositions, and other specific aspects of the catheter fabrication are generally consistent with the catheter designs described hereinbefore.

In a fifth aspect of the present invention, a method for fabricating an elongate catheter having at least two adjacent regions composed of polymeric materials having different mechanical properties comprises vapor depositing a polymeric layer over a joint region where said two axially adjacent regions meet. The polymeric material will have a tensile strength of at least 10 MPa, typically being any one of the polymeric materials set forth above. The polymeric layer will be deposited to a thickness of at least 25 nm, usually being at least 1 μm, preferably being less than 100 μm, more preferably being less than 50 μm, and often being 10 μm or less. The natures, dimensions, and other aspects of the various catheter components have been described previously.

### BRIEF DESCRIPTION OF THE DRAWINGS

FIG. 1 is a cross-sectional view of a portion of a first catheter constructed in accordance with the principles of the present invention.

FIGS. 2A through 2D illustrate the fabrication of a second catheter having a guidewire lumen in a distal tip in accordance with the principles of the present invention.

FIG. 3 illustrates the catheter of FIGS. 2A-2D in use over a guidewire.

FIG. 4 illustrates a third catheter having an injection needle and a guidewire lumen in its distal tip constructed in accordance with the principles of the present invention.

FIG. 5 is a detailed view taken along line 5-5 of FIG. 4.

### DETAILED DESCRIPTION OF THE INVENTION

The present invention provides improved catheter assemblies and methods for their fabrication. The catheters will comprise a tubular catheter body including at least two axially adjacent segments, where those segments have different mechanical properties, such as different hardnesses, different bendabilities, different densities, different elasticities, or the like. As discussed above, the joining of such axially adjacent segments having different mechanical properties can be problematic as the joints therebetween will be subjected to relatively high stresses and kinking as the catheters are advanced through tortuous regions of the anatomy, particularly tortuous regions of the vasculature, such as in the coronary vasculature.

The axially adjacent segments may be joined or unjoined prior to encapsulation by the present invention, as described in more detail herein below. That is, the segments may be brought together in a simple butt or end-to-end joint without the use of any adhesive or other joining techniques other than the encapsulation according to present invention. Usually, however, the segments will be joined by one or more conventional fabrication techniques, such the use of adhesives, heat welding, ultrasonic welding, solvent welding, or the like, prior to encapsulation according to the present invention. Optionally, other joint geometries may be utilized, such as press-fit joints, snap joints, interference joints, or the like.

In all cases, after the axially adjacent segments are brought together, they will be encapsulated in an encapsulation layer which provides for or enhances the tensile strength of the joint. The layer will also help increase kink resistance to reduce the likelihood that the joint will collapse and kink as the catheter is bent during advancement through the target body lumen. The encapsulation layer will usually comprise a vapor deposited polymer, preferably being parylene, silicone, PTFE, PVDF, or the like. Most preferred is parylene vapor deposited over at least the junction region of the catheter to a thickness in the range of 25 nm to 100 μm, preferably 1 μm to 100 μm, and more preferably 5 μm to 50 μm. The parylene or other polymer will be deposited over at least the junction region(s), usually overlapping a junction by at least 0.5 cm on each side, preferably at least 2 cm on each side, and sometimes extending over the entire catheter body. In addition to vapor deposition, some polymers, such as silicones, may be applied by dip-coating or other conventional techniques.

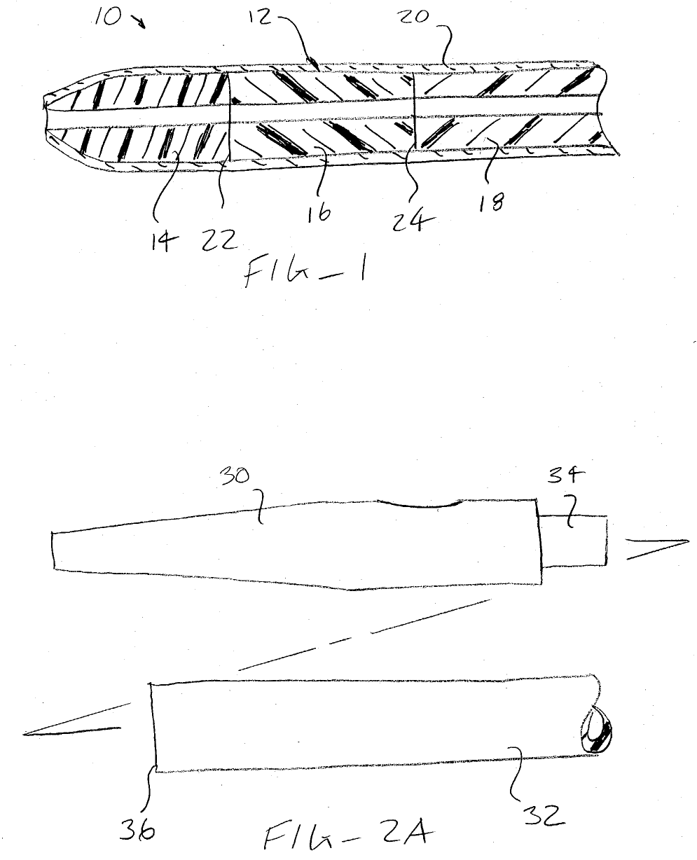

Referring now to FIG. 1, a catheter 10 constructed in accordance with the principles of the present invention comprises a catheter body 12 including three axially adjacent segments 14, 16, and 18. Axially adjacent segment pairs 14/16 and 16/18 are brought together in butt joints which may or may not be attached using an adhesive or other face-to-face attachment approaches. The adjacent segments 14, 16, and 18 are joined by encapsulation layer 20 which extends over both junction regions 22 and 24 as well as over the entire distal end of the catheter.

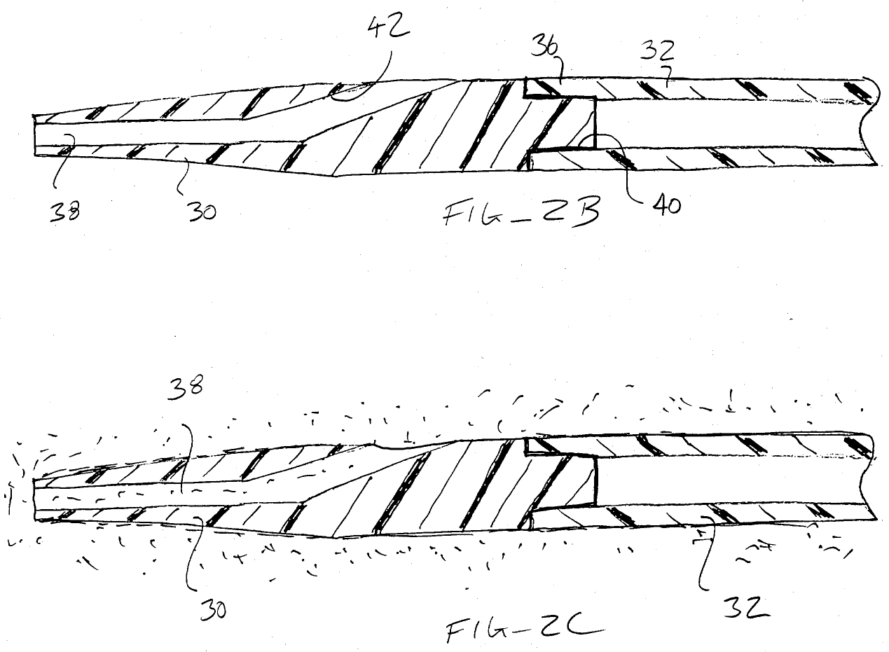

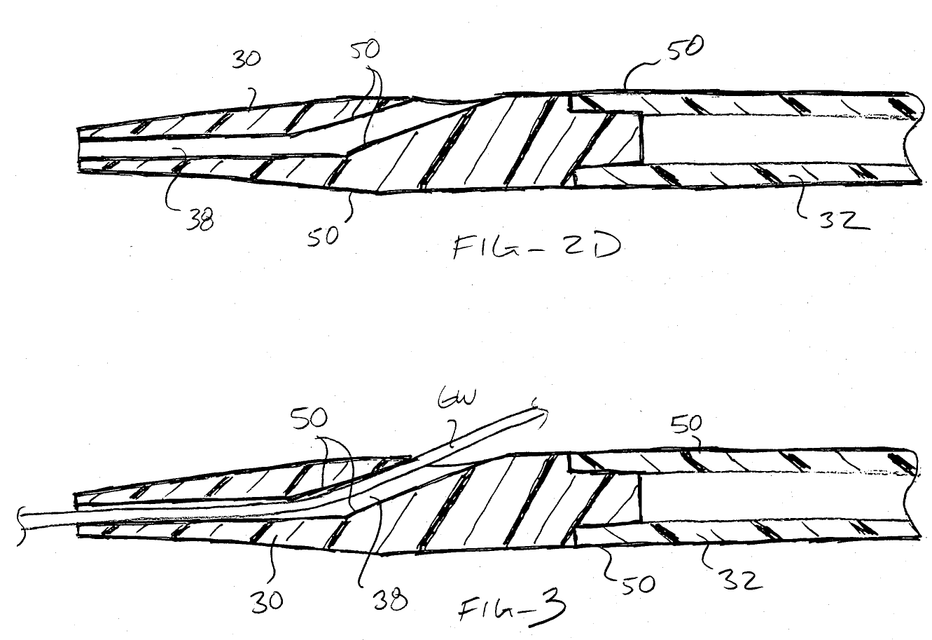

Fabrication of a rapid exchange catheter in accordance with the principles of the present invention is illustrated in FIGS. 2A-2D. Initially, as shown in FIG. 2A, a catheter tip 30 and catheter body 32 are provided. The catheter tip has a male fitting 34 which extends into a recession within a distal end 36 of the catheter body 32. The tip 30 is press-fit into the distal end 36 of the catheter body 32, as shown in FIG. 2B. Preferably, a layer of adhesive 40 is provided in order to initially join the tip 30 to the body 32.

In order to provide an encapsulation layer in accordance with the present invention, the assembly of catheter tip 30 and catheter body 32 is placed in a chamber for vapor deposition of parylene onto all exposed surfaces, including the interior surface 42 of guidewire lumen 38 and the tip 30. Vapor deposition of parylene may be accomplished in conventional equipment, such as Parylene Deposition Systems, commercially available from Cookson Electronics Equipment, Specialty Coating Systems, Indianapolis, Ind., USA. Parylene dimer is vaporized at about 130° C., cleaved into monomer form at 690° C. and deposited at room temperature onto a catheter body substrate under a vacuum of 25 mTorr.

After the vapor deposition process is complete, the catheter is coated with a parylene encapsulation layer 50 over all exposed surfaces, including the interior surfaces of the guidewire lumen 38, shown in FIG. 2. The parylene layer 50 over the exterior of the catheter, particularly that over the junction between the tip 30 and the catheter body 32, both enhances the tensile strength of the joint therebetween and reduces the likelihood that the catheter will kink at this joint. In addition, parylene coating of the guidewire lumen 38 enhances lubricity of that lumen when introduced over a guidewire GW as shown in FIG. 3.

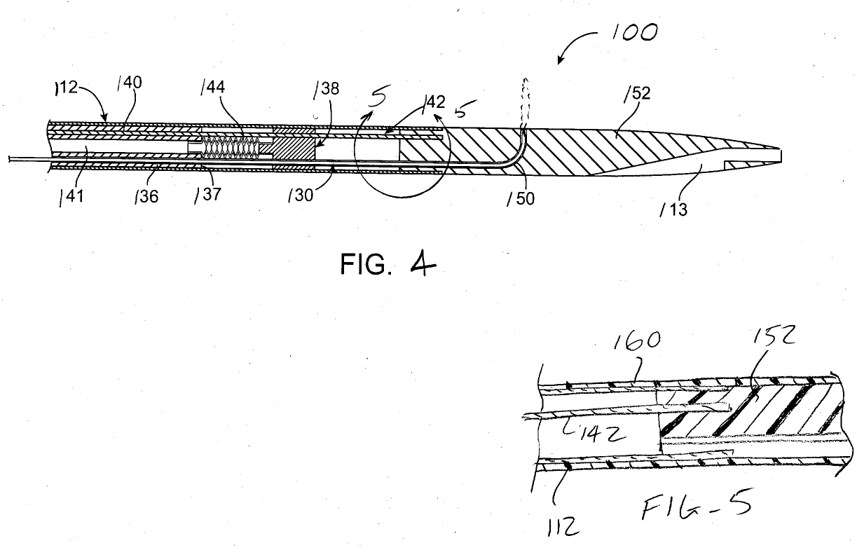

Referring now to FIGS. 4 and 5, a needle injection catheter 100 is illustrated. The construction of the catheter 100 is generally taught in co-pending provisional application No. ______, (Attorney Docket No. 021621-001500US), filed on Mar. 19, 2003, full disclosure of which is incorporated herein by reference. The needle injection catheter 100 comprises a catheter body 112 having a distal end 114 and a proximal 116. Usually, a guide wire lumen 113 will be provided in a distal nose 152 of the catheter, although over-the-wire and embodiments which do not require guide wire placement will also be within the scope of the present invention. A two-port hub (not shown) is attached to the proximal end 116 of the catheter body 112 and includes a first port for delivery of a hydraulic fluid, e.g., using a syringe, and a second port for delivering a pharmaceutical agent, e.g., using a syringe. A reciprocatable, deflectable needle 130 is mounted near the distal end of the catheter body 112 and is shown in its laterally advanced in broken line.

The distal end 114 of the catheter body 112 has a main lumen 136 which holds the needle 130, a reciprocatable piston 138, and a hydraulic fluid delivery tube 140. The piston 138 is mounted to slide over a rail 142 and is fixedly attached to the needle 130. Thus, by delivering a pressurized hydraulic fluid through a lumen 141 tube 140 into a bellows structure 144, the piston 138 may be advanced axially toward the distal tip in order to cause the needle to pass through a deflection path 150 formed in a catheter nose 152.

As best shown in FIG. 5, a junction region between the catheter body 112 and the catheter nose 152 is reinforced with an encapsulation layer 160 in accordance with the principles of the present invention. Encapsulation layer 160 may be composed of any of the materials set forth above and will generally have the properties of the materials set forth above as well. In particular, the encapsulation layer 160 is preferably vapor deposited parylene having a tensile strength in the axial direction of at least 10 MPa.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A catheter comprising:
a tubular catheter body comprising at least two axially adjacent segments composed of materials having different mechanical properties; and
an encapsulation layer covering a junction region where the two segments meet, wherein the encapsulation layer has a tensile strength of at least 10 MPa.

2. A catheter as in claim 1, wherein one segment is harder than another segment.

3. A catheter as in claim 2, wherein the segments are joined in a butt joint.

4. A catheter as in claim 3, wherein one segment comprises a proximal body and the other segment comprises a distal tip.

5. A catheter as in claim 4, wherein the distal tip comprises a guidewire lumen.

6. A catheter as in claim 5, wherein the guidewire lumen has a length of 5 cm or less.

7. A catheter as in claim 1, wherein the encapsulation layer comprises a polymer.

8. A catheter as in claim 7, wherein the polymer is vapor deposited.

9. A catheter as in claim 8, wherein the vapor deposited polymer has an average thickness of 100 μm or below.

10. A catheter as in claim 9, wherein the polymer is selected from the group consisting of parylene, polyethylene, silicone, polytetrafluoroethylene, and polyvinylidene fluoride.

11. A rapid exchange catheter comprising:
a catheter body; and
a tip disposed of a distal end of the catheter body, wherein the tip has a guidewire lumen having a length of 5 cm or less;
wherein the tip is joined to the catheter body by an encapsulation layer having a tensile strength of at least 10 MPa.

12. A rapid exchange catheter as in claim 11, wherein the encapsulation layer extends at least partially over the surface of the guidewire lumen.

13. A rapid exchange catheter as in claim 12, wherein the encapsulation layer is lubricious and extends over at least most of the guidewire lumen surface.

14. A rapid exchange catheter as in claim 11, wherein the encapsulation layer comprises a polymer.

15. A rapid exchange catheter as in claim 14, wherein the polymer is vapor deposited.

16. A rapid exchange catheter as in claim 15, wherein the vapor deposited polymer has an average thickness of 100 μm or below.

17. A rapid exchange catheter as in claim 16, wherein the polymer is selected from the group consisting of parylene, polyethylene, silicone, polytetrafluoroethylene, and polyvinylidene fluoride.

18. A rapid exchange catheter as in claim 11, wherein the tip is harder than the catheter body.

19. A rapid exchange retractor as in claim 11, wherein the catheter body is harder than the tip.

20. A rapid exchange catheter as in claim 11, wherein the catheter tip or catheter body are joined in a butt joint.

21. A needle injection catheter comprising:
a catheter body having a proximal end and a distal end;
a needle reciprocatably disposed in the catheter body near the distal end; and
wherein a guidewire lumen is disposed in the distal end of the catheter body distally of the needle.

22. A needle injection catheter as in claim 21, wherein the needle advances outwardly through the catheter body in a first radial direction, wherein the guidewire lumen is circumferentially spaced-apart from the needle.

23. A needle injection catheter as in claim 22, consisting essentially of a single reciprocatable needle, wherein the guidewire lumen is offset by at least 90°.

24. A needle injection catheter as in claim 23, wherein the guidewire lumen is offset by at least 135°.

25. A needle injection catheter as in claim 21, wherein the catheter body comprises at least a proximal body portion and a distal tip, wherein said proximal body portion and tip are composed of materials having different mechanical properties and are joined at a junction region.

26. A needle injection catheter as in claim 25, further comprising an encapsulation layer covering the junction region.

27. A needle injection catheter as in claim 21, wherein the encapsulation layer comprises a polymer.

28. A needle injection catheter as in claim 27, wherein the polymer is vapor deposited.

29. A needle injection catheter as in claim 28, wherein the vapor deposited polymer has an average thickness of 100 μm or below.

30. A needle injection catheter as in claim 24, wherein the polymer is selected from the group consisting of parylene, polyethylene, silicone, polytetrafluoroethylene, and polyvinylidene fluoride.

31. A needle injection catheter as in claim 21, wherein the tip is harder than the proximal body portion.

32. A needle injection catheter as in claim 21, wherein the proximal body portion is harder than the tip.

33. A needle injection catheter as in claim 21, wherein the segments are joined in a butt joint.

34. A needle injection catheter as in claim 21, wherein the distal tip comprises a guidewire lumen.

35. A needle injection catheter as in claim 34, wherein the guidewire lumen has a length of 5 cm or less.

36. A method for fabricating an elongate catheter having at least two segments adjacent in an axial direction composed of polymeric materials having different mechanical properties, said method comprising;
encapsulating a joint region where said two adjacent segments meet with a layer of material having a tensile strength in the axial direction of at least 10 MPa.

37. A method as in claim 36, wherein encapsulating comprises vapor depositing a polymer.

38. A method as in claim 37, wherein the polymer is selected from the group consisting of parylene, polyethylene, silicone, polytetrafluoroethylene, and polyvinylidene fluoride.

39. A method as in claim 36, wherein the material is deposited to a thickness of 100 μm or below.

40. A method as in claim 36, wherein one segment is harder than another segment.

41. A method as in claim 40, wherein the segments are joined in a butt joint.

42. A method as in claim 41, wherein one segment comprises a proximal body and the other segment comprises a distal tip.

43. A method as in claim 42, wherein the distal tip comprises a guidewire lumen.

44. A method as in claim 43, wherein the guidewire lumen has a length of 5 cm or less.

45. A method as in claim 44, wherein the encapsulating material covers at least a portion of the guidewire lumen.

46. A method for fabricating an elongate catheter having at least two regions adjacent in an axial direction composed of polymeric materials having different mechanical properties, said method comprising:
vapor depositing a polymeric layer over a joint region where said two adjacent regions meet.

47. A method as in claim 46, wherein the layer of polymeric material has a tensile strength of at least 10 MPa.

48. A method as in claim 46, wherein the polymeric layer is a material selected from the group consisting of parylene, polyethylene, silicone, polytetrafluoroethylene, and polyvinylidene fluoride.

49. A method as in claim 46, wherein the polymeric layer is deposited to a thickness of at least 25 nm.

50. A method as in claim 36, wherein one segment is harder than another segment.

51. A method as in claim 50, wherein the segments are joined in a butt joint.

52. A method as in claim 51, wherein one segment comprises a proximal body and the other segment comprises a distal tip.

53. A method as in claim 52, wherein the distal tip comprises a guidewire lumen.

54. A method as in claim 53, wherein the guidewire lumen has a length of 5 cm or less.

55. A method as in claim 54, wherein the polymer is deposited over at least a portion of the guidewire lumen.
